(19) United States
(12) Patent Application Publication
(10) Pub. No.: US 2026/0174485 A1
(43) Pub. Date: Jun. 25, 2026

(54) MEDICAL BONE SCREW

(71) Applicants: Hyung Joo Park, Seoul (KR); Gi Ae Oh, Seoul (KR); Kyu Won Park, Seoul (KR); Kyu Min Park, Seoul (KR)

(72) Inventor: Hyung Joo Park, Seoul (KR)

(73) Assignees: Hyung Joo Park, Seoul (KR); Gi Ae Oh, Seoul (KR); Kyu Won Park, Seoul (KR); Kyu Min Park, Seoul (KR)

(21) Appl. No.: 19/211,151

(22) Filed: May 17, 2025

(30) Foreign Application Priority Data
Dec. 23, 2024 (KR) 10-2024-0194694
Mar. 20, 2025 (KR) 10-2025-0035800

(51) Int. Cl. A61B 17/86

(52) U.S. Cl. CPC A61B 17/8615; A61B 17/8635

(57) ABSTRACT

A medical bone screw with a new structure capable of improving fixation strength with human tissue such as bone tissue and minimizing damage to human tissue during insertion is disclosed. The medical bone screw includes a head, a body connected to the head and having a spiral shape with dimensions appropriate for medical bone screw applications, and a central space defined by the body.

### CROSS-REFERENCE TO RELATED APPLICATIONS

This application claims the priority of Korean Patent Application Nos. 10-2024-0194694 filed on Dec. 23, 2024, and 10-2025-0035800 filed on Mar. 20, 2025, in the Korean Intellectual Property Office, the disclosures of which are incorporated herein by reference.

### BACKGROUND OF THE DISCLOSURE

### Technical Field

The present invention relates to a medical bone screw, and more particularly, to a medical bone screw capable of improving fixation strength with human tissue and minimizing damage to human tissue.

### Background Art

Generally, bone screws are essential medical devices used in various medical fields such as orthopedics, neurosurgery, and dentistry.

For example, in general fracture treatment, a bone fixation device is used to treat fracture patients by incising the soft tissue of the fracture patient, supporting a plate on the surface of the fractured bone, and fixing it with screws.

In the field of spine surgery, screws and reinforcement rods are installed to connect and fix between vertebral bodies to strengthen the vertebrae through treatment methods such as Spinal Fusion, Spinal Deformity Correction, and Spinal Fracture Treatment.

In addition, in the field of dentistry, dental implant procedures for treating tooth defects are widely performed, in which screws are inserted and fixed into the maxillary or mandibular bone, and then artificial teeth are implanted on top of them.

However, conventional bone screws have weak fixation strength with bone tissue, frequently causing loosening phenomenon (Screw Loosening) after the procedure. This phenomenon can cause problems such as bone remodeling, inflammation, screw migration, and delayed healing.

Additionally, conventional bone screws have the problem of forming holes in the bone tissue corresponding to the diameter of the screw when inserted into the bone. This causes unnecessary damage to bone tissue, which can lead to side effects such as prolonged healing periods or reduced fixation strength.

Therefore, there is a demand for the development of a new structure of bone screw that can improve fixation strength with bone tissue and minimize damage to bone tissue during insertion.

### SUMMARY OF THE DISCLOSURE

A feature to be achieved by the present disclosure is to provide a medical bone screw with a new structure that can improve fixation strength with human tissue such as bone tissue and minimize damage to human tissue during insertion.

A medical bone screw according to an embodiment of the present invention includes: a head; a body connected to the head and having a spiral shape with dimensions appropriate for medical bone screw applications; and a central space defined by the body.

A medical bone screw according to another embodiment of the present invention includes: a head that can be coupled with a medical tool; and an anchor unit having a first portion extending from the head, and a second portion having a spiral shape extending from the first portion to be inserted into a patient's bone to accommodate bone tissue.

A medical medium for human tissue attachment according to an embodiment of the present invention includes: a body having an open spiral shape to penetrate human tissue; and a tissue accommodation space defined by the open spiral shape and configured to improve fixation strength with respect to the human tissue attachment when compared to a medical medium that lacks the tissue accommodation space.

The medical bone screw according to the present invention effectively distributes stress within bone tissue through a spiral-shaped body, and unlike conventional linear bone screws, it evenly distributes load throughout the entire body, preventing stress concentration in specific areas. Therefore, it has the effect of reducing the risk of damage or injury when inserted into human tissue such as bone tissue, extending lifespan, and preventing destruction of human tissue.

Additionally, the medical bone screw according to the present invention has excellent fixation strength as the contact area with human tissue such as bone tissue is increased compared to conventional bone screws.

Furthermore, the medical bone screw according to the present invention allows minimally invasive insertion, minimizes damage to human tissue such as bone tissue, and can shorten recovery time.

Moreover, the medical bone screw of the present invention can prevent loosening phenomena that may occur after the procedure and secure long-term stability.

The features of the present disclosure are not limited to the aforementioned features, and other objects, which are not mentioned above, will be understood by a person having ordinary skill in the art from the following description.

### BRIEF DESCRIPTION OF THE DRAWINGS

The above and other aspects, features and other characteristics of the present invention embodiments will be more clearly understood from the following detailed description taken in conjunction with the accompanying drawings, in which:

FIG. 1 is a front view showing a medical bone screw according to an embodiment of the present invention;

FIG. 2 is a front view showing a medical bone screw according to another embodiment of the present invention;

FIG. 3 shows the medical bone screw shown in FIG. 2 coupled with a medical tool;

FIG. 4 is a front view showing a medical bone screw according to yet another embodiment of the present invention;

FIG. 5 is a front view showing a medical bone screw according to yet another embodiment of the present invention;

FIG. 6 is a front view showing a medical bone screw according to yet another embodiment of the present invention;

FIG. 7 is a bottom view showing the medical bone screw shown in FIG. 6;

FIG. 8 schematically shows the medical bone screw shown in FIG. 6 inserted into bone tissue;

FIG. 9 schematically shows the medical bone screw shown in FIG. 6 separated from bone tissue;

FIG. 10 is a front view showing a medical bone screw according to yet another embodiment of the present invention;

FIG. 11 is a front view showing a medical bone screw according to yet another embodiment of the present invention;

FIG. 12 is a front view showing a medical bone screw according to yet another embodiment of the present invention;

FIG. 13 is a front view showing a medical bone screw according to yet another embodiment of the present invention;

FIG. 14 is a front view showing a medical bone screw according to yet another embodiment of the present invention; and

FIG. 15 is a front view showing a conventional bone screw.

### DETAILED DESCRIPTION OF THE EMBODIMENT(S)

The advantages and features of this specification, and methods of achieving them will become apparent with reference to embodiments described in detail later with the accompanying drawings. However, this specification is not limited to the embodiments disclosed hereinafter but will be implemented in various different forms, and the embodiments are provided merely to make the disclosure of this specification complete and to fully convey the scope of the specification to those skilled in the art to which this specification pertains.

The shapes, areas, ratios, angles, numbers, etc. disclosed in the drawings for describing embodiments of this specification are exemplary, so this specification is not limited to the illustrated matters. Throughout the specification, the same reference numerals refer to the same components. Also, in explaining this specification, detailed descriptions of well-known related technologies may be omitted if they are deemed to unnecessarily obscure the gist of this specification. When terms such as ‘includes’, ‘has’, ‘consists of’, etc. are used in this specification, other parts may be added unless ‘only’ is used. When a component is expressed in the singular, it includes the plural case unless there is a specific explicit statement.

When interpreting components, even without explicit statement, it is interpreted to include an error range.

In case of descriptions of positional relationships, for example, when the positional relationship between two parts is described as ‘on’, ‘on top of’, ‘below’, ‘beside’, etc., unless ‘directly’ is used, one or more other parts may be positioned between the two parts.

Further, terms such as first, second, etc. are used to describe various components, but these components are not limited by these terms. These terms are only used to distinguish one component from another component.

The same reference numerals refer to the same components throughout the specification.

The area and thickness of each structure shown in the drawings are shown for convenience of explanation, and this specification is not necessarily limited to the area and thickness of the structure shown.

In the embodiments, a ‘module’ or ‘unit’ performs at least one function or operation and may be implemented in hardware or software, or a combination of hardware and software. Also, a plurality of ‘modules’ or a plurality of ‘units’ may be integrated into at least one module except for ‘modules’ or ‘units’ that need to be implemented in specific hardware.

The features of the various embodiments of this specification may be partially or completely combined or combined with each other, technically possible in various interlocking and operation, and the embodiments may be implemented independently of each other or may be implemented together in an associated relationship.

Hereinafter, the present invention will be described with reference to the drawings.

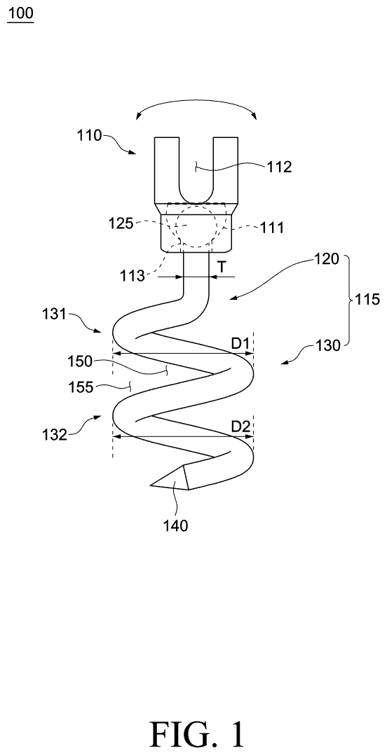

FIG. 1 is a front view showing a medical bone screw according to an embodiment of the present invention.

The medical bone screw 100 according to an embodiment of the present invention includes a head 110, an anchor unit 115 connected to the head 110, and a tip 140 disposed at the end of the anchor unit 115.

The medical bone screw 100 according to an embodiment of the present invention is a medical medium that can fix or connect human tissue by being inserted into human tissue such as bone tissue. Various medical tools, medical connecting members, or medical implants can be connected to the medical bone screw 100. Also, the medical bone screw 100 can be inserted into human tissue or separated from human tissue by medical tools such as medical drivers.

The head 110 is a part that is not inserted into bone tissue. The head 110 is configured to allow a medical connecting member to be coupled. The head 110 includes a head groove 111, an outwardly open coupling groove 112, and a through hole 113 into which a part of the anchor unit 115 is inserted. The head groove 111 and the through hole 113 are connected, and a part of the anchor unit 115 is inserted into the head groove 111 and the through hole 113. A medical connecting member or medical implant can be coupled to the head 110 by inserting the medical connecting member or medical implant into the coupling groove 112. The head 110 is tiltably coupled to the anchor unit 115.

According to an embodiment of the present invention, the medical bone screw 100 allows a medical connecting member or medical implant to be angle-adjustably connected to the anchor unit 115 fixed to human tissue such as bone tissue through the head 110.

Therefore, the angle of the medical connecting member or medical implant can be optimized according to the anatomical structure of the surgical site or the patient's condition. In particular, the tilting function of the head 110 allows the angle of the medical connecting member or implant to be freely adjusted even after the medical bone screw 100 is fixed to the bone tissue, thereby improving the accuracy of the surgery and the convenience of the procedure.

Additionally, the tiltable coupling structure of the head 110 enables fine angle adjustments according to the patient's movement or load changes after surgery, providing long-term treatment stability, and is particularly useful in procedures requiring precise angle adjustment, such as spinal correction or dental implants.

The specific configuration of the head 110 can be variously changed.

The anchor unit 115 includes a first portion 120 and a second portion 130 extending from the first portion 120. The first portion 120 extends from the head 110 to connect the head 110 and the second portion 130. The first portion 120 has a straight shape and extends from the head 110. The second portion 130 has a spiral shape to be inserted into the patient's bone to accommodate bone tissue.

Hereinafter, the first portion will be referred to as the neck, and the second portion will be referred to as the body.

The neck 120 is positioned between the head 110 and the body 130, connecting them. The neck 120 has a straight shape. This type of neck 120 can more stably transfer rotational force delivered from medical tools such as medical drivers to the body 130. However, the shape of the neck 120 can be variously modified.

The cross-sectional shape of the neck 120 can be circular, but is not limited thereto.

The connection area between the neck 120 and the body 130 can be curved to prevent stress concentration.

A portion of the neck 120 is inserted into the through hole 113 of the head 110. A head joint 125 that is positioned in the head groove 111 is connected to the end of the neck 120. The head joint 125 has a size that cannot pass through the through hole 113. The head 110 is tiltably coupled to the head joint 125.

The body 130 is the part that is inserted into the patient's bone. The body 130 has an open spiral shape and can accommodate bone tissue. The body 130 can have a form that winds in a clockwise or counterclockwise direction.

The body 130 includes a plurality of spirals 131, 132 connected in sequence. Each spiral 131, 132 can have a form that winds in a clockwise or counterclockwise direction.

The plurality of spirals 131, 132 includes a first spiral 131 connected to the neck 120 and a second spiral 132 connected to the first spiral 131. The tip 140 is positioned at the end of the second spiral 132. The first spiral 131 and the second spiral 132 can be smoothly surface-treated considering direct contact with bone tissue.

The cross-sectional shape of the first spiral 131 and the second spiral 132 can be circular like the neck 120, but is not limited thereto. The first diameter D1 of the first spiral 131 and the second diameter D2 of the second spiral 132 can be the same, but are not limited thereto. And the pitch of the first spiral 131 and the pitch of the second spiral 132 can be the same, but are not limited thereto.

Additionally, the first spiral 131 and the second spiral 132 can be made with the same thickness, but are not limited thereto. The thickness of the first spiral 131 and the second spiral 132 can be the same as the thickness T of the neck 120, but is not limited thereto.

The tip 140 is positioned at the end of the second spiral 132. The tip 140 forms a spiral shape with the body 130. The tip 140 extends from the end of the second spiral 132 in the winding direction of the second spiral 132. The tip 140 has a pointed shape where the thickness gradually decreases as it gets farther from the end of the second spiral 132 to penetrate bone tissue. The tip 140 first penetrates the bone tissue at the end of the body 130 and enters into the bone tissue, forming a passage for the body 130 to enter the bone tissue.

The tip 140 can be made in a non-cutting edge form. That is, the tip 140 has a smooth curved surface without a cutting edge. This non-cutting edge structure of the tip 140 has the characteristic of being inserted by pushing the bone tissue aside rather than cutting it. Therefore, it can minimize damage to bone tissue and promote bone tissue healing.

The specific structure of the tip 140 can be variously modified. That is, the tip 140 can be made with a cutting edge structure. Also, depending on the human tissue to which the medical bone screw 100 is applied, the tip 140 may have shapes other than a pointed shape where the thickness gradually decreases as it gets farther from the end.

The pointed tip 140 can be omitted if necessary.

The medical bone screw 100 according to an embodiment of the present invention includes a central space 150 and an intermediate space 155 defined by the body 130. The central space 150 can be positioned inside the body 130 in a cylindrical shape. The intermediate space 155 is positioned outside the central space 150. The central space 150 and the intermediate space 155 form a tissue accommodation space that can accommodate human tissue such as bone tissue.

Because the body 130 is made in a spiral shape with an empty inside, the elasticity of the medical bone screw 100 is increased. This elasticity of the medical bone screw 100 can absorb fine loads or impacts and alleviate stress concentration that may occur at the interface with bone tissue.

The medical bone screw 100 according to an embodiment of the present invention can be manufactured with biocompatible materials such as medical metal materials. Therefore, the medical bone screw 100 according to an embodiment of the present invention ensures high strength and durability, and due to its excellent biocompatibility, it can be maintained stably in the human body for a long period. Additionally, the surface of the medical bone screw 100 according to an embodiment of the present invention can be smoothly treated considering direct contact with bone tissue, minimizing damage to surrounding tissues.

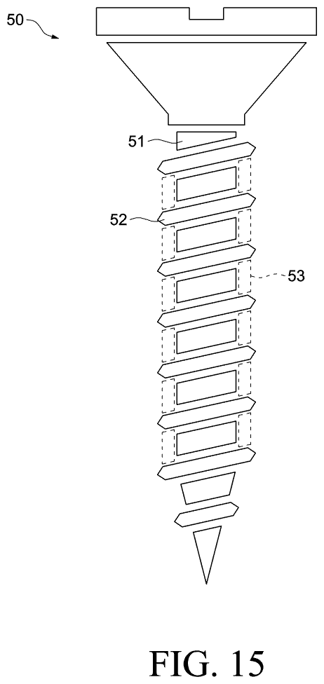

The medical bone screw 100 according to an embodiment of the present invention has several advantages compared to the conventional bone screw 50 shown in FIG. 15, as the body 130 inserted into bone tissue 20 has a spiral shape.

The conventional bone screw 50 consists of a core 51 in the center and threads 52 wrapping around it. The conventional product forms a large hole as big as the core 51 diameter as it destroys bone tissue when inserted. Also, the bonding strength is mainly maintained by the pressure applied by the threads 52 to the bone tissue and the friction between the bone tissue and the threads 52, so only a limited amount of bone tissue is located in the space 53 between the threads 52, resulting in a small contact area and poor stability.

In contrast, the medical bone screw 100 of the present invention has a body 130 made in a coil shape with an empty inside, so when inserted into bone tissue, the body 130 rotates into the bone tissue, creating only a small hole as thick as the body 130. At this time, bone tissue naturally flows into the central space 150 inside the spirals 131, 132 and the intermediate space 155 between the spirals 131, 132, minimizing damage to bone tissue. And as bone tissue flows into the central space 150 and the intermediate space 155, the bonding area greatly increases, and the contact area between the spirals 131, 132 and bone tissue becomes relatively larger, allowing for more solid bonding.

Additionally, the medical bone screw 100 of the present invention provides significantly improved pull-out strength compared to conventional products. In tensile strength tests on bone tissue, conventional products showed an average pull-out strength of 339.95N, while the present invention product showed an average pull-out strength of 493.71N. This means about 45% improved pull-out strength, and was confirmed to be statistically significant by T-test (p-value=0.007). These objective results prove that the present invention product substantially improves bonding strength with bone tissue.

This improved pull-out strength stems from the unique structural features of the medical bone screw 100 of the present invention. The coil-shaped body 130 with an empty inside maximizes the contact area by allowing bone tissue to flow into the central space 150 inside the spirals 131, 132 and the intermediate space 155 between the spirals 131, 132. Also, these structural characteristics evenly distribute loads, greatly improving resistance to tensile forces. Unlike conventional products that maintain fixation strength only through contact with bone tissue located in the core 51 and limited space 53, the medical bone screw 100 of the present invention provides more uniform and strong fixation strength as bone tissue naturally fills and bonds with the wide central space 150 and intermediate space 155.

This improved pull-out strength has very important clinical implications. Loosening of bone screws is one of the main causes of surgical failure, and the medical bone screw 100 of the present invention can greatly reduce this risk. In particular, it has the advantage of expanding the clinical application range by providing stable fixation strength even in osteoporosis patients or areas with weak bone quality.

The medical bone screw 100 of the present invention can effectively distribute stress within bone tissue. Unlike conventional linear bone screws 50, it evenly distributes load throughout the entire body 130, preventing stress concentration in specific areas, thereby extending lifespan and preventing bone tissue destruction.

Additionally, the medical bone screw 100 of the present invention allows for minimally invasive insertion compared to conventional bone screws 50, thereby minimizing damage to bone tissue and shortening recovery time.

Furthermore, the medical bone screw 100 of the present invention ensures long-term stability through improved pull-out strength, reduces the need for reoperation, and can be effectively applied to various patient groups.

The medical bone screw 100 of the present invention can be utilized in various medical fields. In the field of neurosurgery, it can be applied as a spinal fixation screw for spinal fusion surgery or spinal correction procedures, and in the field of orthopedics, it can be used as a fracture treatment screw for fixing fractures of iliac bones or limb bones. Additionally, in the field of dentistry, it can be utilized as an implant treatment screw or orthodontic screw.

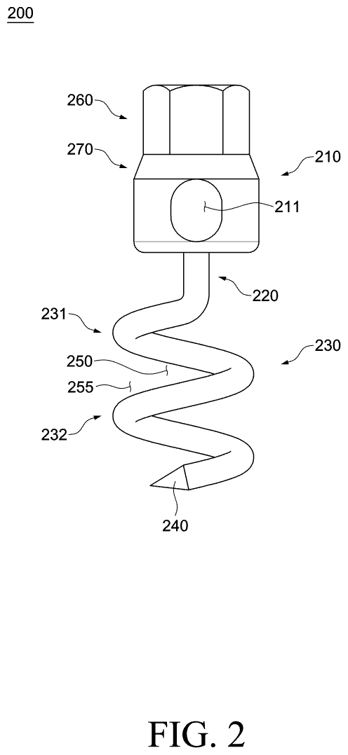

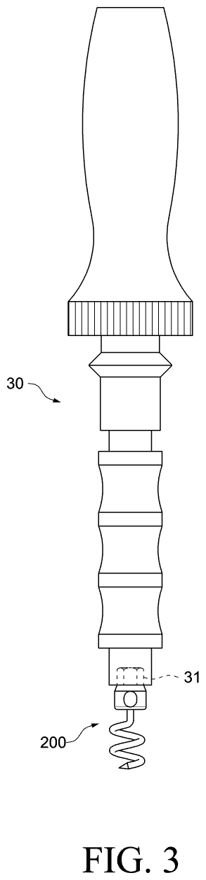

FIG. 2 is a front view showing a medical bone screw according to another embodiment of the present invention. FIG. 3 shows the medical bone screw shown in FIG. 2 coupled with a medical tool.

As shown in the drawings, the medical bone screw 200 according to another embodiment of the present invention includes a head 210, a neck 220 connected to the head 210, a body 230 connected to the neck 220, a tip 240 disposed at the end of the body 230, a central space 250 and an intermediate space 255 defined by the body 230, and a head protrusion 260 protruding from the head 210. The neck 220 and the body 230 constitute an anchor unit.

The head 210 includes a coupling hole 211 to which medical tools such as medical wires or medical connecting members can be connected.

The body 230 includes a plurality of spirals 231, 232 connected in sequence. The plurality of spirals 231, 232 includes a first spiral 231 connected to the neck 220 and a second spiral 232 connected to the first spiral 231.

The tip 240 is positioned at the end of the second spiral 232.

The head protrusion 260 protrudes from the head 210 to be detachably coupled with a medical tool 30 such as a medical driver. The head protrusion 260 can be made in a shape corresponding to the shape of the engagement groove 31 positioned at the end of the medical tool 30. For example, the head protrusion 260 can be made in a hexagonal column shape. As the head protrusion 260 is stably inserted into the engagement groove 31 of the medical tool 30, precise rotational control is possible by minimizing play in the rotational direction of the medical tool 30. As shown, the thickness of the head protrusion 260 can be smaller than the thickness of the head 210, but is not limited thereto.

A connecting body 270 is positioned between the head 210 and the head protrusion 260. The connecting body 270 can be made in a cylindrical shape where the diameter gradually decreases from the head 210 toward the head protrusion 260.

The gradually decreasing diameter structure of the connecting body 270 serves to uniformly distribute the rotational force transmitted from the medical tool 30 to the head 210. This structure enhances the structural stability of the medical bone screw 200 by preventing stress concentration that may occur when excessive rotational force is applied.

The medical bone screw 200 according to another embodiment of the present invention can be stably coupled with the medical tool 30 as it includes the head protrusion 260 that is detachably inserted into the engagement groove 31 of the medical tool 30. And the rotational force of the medical tool 30 can be stably transmitted to the head 210 through the head protrusion 260. Therefore, it can be more stably inserted into bone tissue 20 by the medical tool 30, and can be more smoothly separated from bone tissue 20 by the medical tool 30.

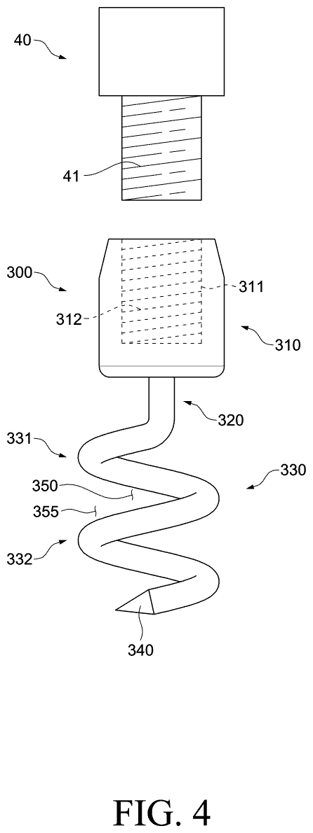

FIG. 4 is a front view showing a medical bone screw according to yet another embodiment of the present invention.

As shown in the drawing, the medical bone screw 300 according to yet another embodiment of the present invention includes a head 310, a neck 320 connected to the head 310, a body 330 connected to the neck 320, a tip 340 disposed at the end of the body 330, and a central space 350 and an intermediate space 355 defined by the body 330. The neck 320 and the body 330 constitute an anchor unit.

The head 310 includes a coupling groove 311 and a head thread 312 disposed in the coupling groove 311. A medical abutment 40 can be inserted into the coupling groove 311. The head thread 312 can engage with an abutment thread 41 of the medical abutment 40.

The body 330 includes a plurality of spirals 331, 332 connected in sequence. The plurality of spirals 331, 332 includes a first spiral 331 connected to the neck 320 and a second spiral 332 connected to the first spiral 331.

The tip 340 is positioned at the end of the second spiral 332.

The medical bone screw 300 according to yet another embodiment of the present invention can stably fix the medical abutment 40 to bone tissue as it includes the coupling groove 311 and the head thread 312.

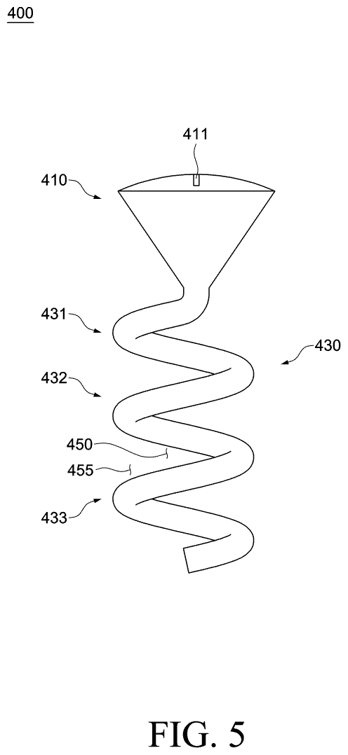

FIG. 5 is a front view showing a medical bone screw according to yet another embodiment of the present invention.

As shown in the drawing, the medical bone screw 400 according to yet another embodiment of the present invention includes a head 410, a body 430 connected to the head 410, and a central space 450 and an intermediate space 455 defined by the body 430.

The head 410 has a recess 411 into which medical tools such as a medical driver can be inserted. A medical tool can engage with the head 410 as the end of the medical tool is inserted into the recess 411.

The body 430 is directly connected to the head 410. The body 430 includes a plurality of spirals 431, 432, 433 connected in sequence. The plurality of spirals 431, 432, 433 includes a first spiral 431 connected to the head 410, a second spiral 432 connected to the first spiral 431, and a third spiral 433 connected to the second spiral 432.

The diameter, pitch, and thickness of each of the first spiral 431, the second spiral 432, and the third spiral 433 can all be the same, but are not limited thereto.

The medical bone screw 400 according to yet another embodiment of the present invention has the head 410 directly connected to the body 430, and does not have a pointed tip. This structure can be applied to medical bone screws according to various embodiments shown in the drawings.

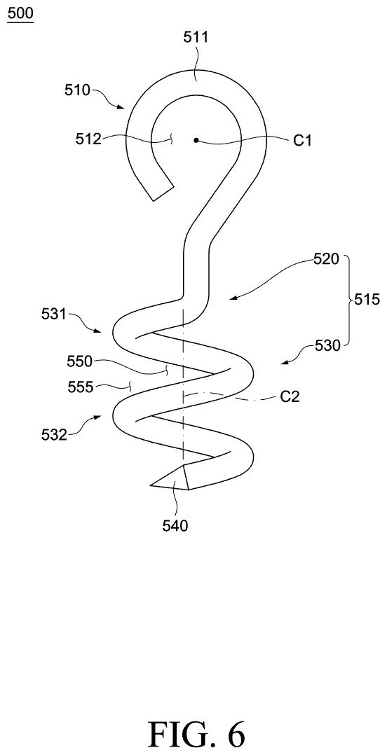

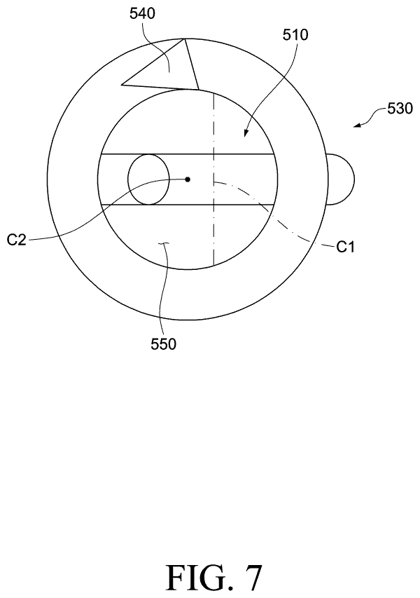

FIG. 6 is a front view showing a medical bone screw according to yet another embodiment of the present invention. FIG. 7 is a bottom view showing the medical bone screw shown in FIG. 6.

As shown in the drawings, the medical bone screw 500 according to yet another embodiment of the present invention includes a head 510, an anchor unit 515 connected to the head 510, and a tip 540 disposed at the end of the anchor unit 515.

The head 510 has a hook shape to allow coupling with a medical tool. A medical tool can be connected to the head 510, or a medical implant can be coupled. In addition, a medical tool for rotating the medical bone screw 500 can be detachably coupled to the head 510.

The head 510 includes a ring 511. A coupling hole 512 is positioned in the ring 511. A medical tool can be connected to the ring 511, or a medical implant can be coupled. The ring 511 has a circular cross-sectional shape. Additionally, the ring 511 has a curved shape to allow medical tools or medical implants to be firmly coupled.

The specific configuration of the head 510 can be variously changed. For example, the ring 511 can be made in shapes other than circular or oval. Also, a fine uneven pattern to increase friction with medical tools can be positioned on the surface of the head 510. In addition, the cross-sectional shape of the ring 511 can be changed to shapes other than circular, such as oval.

The anchor unit 515 includes a neck 520 and a body 530 extending from the neck 520.

The neck 520 is positioned between the head 510 and the body 530, connecting them. The neck 520 has a straight shape.

The cross-sectional shape of the neck 520 can be circular like the cross-sectional shape of the head 510, but is not limited thereto.

The connection area between the neck 520 and the body 530 can be curved to prevent stress concentration.

The body 530 is the part that is inserted into the patient's bone. The body 530 can be inserted into the patient's bone to accommodate bone tissue. The body 530 has a spiral shape. The body 530 can have a form that winds in a clockwise or counterclockwise direction.

The winding direction of the body 530 and the winding direction of the ring 511 of the head 510 are different. That is, the central axis C1 of the coupling hole 512 of the head 510 and the central axis C2 of the spiral shape are substantially perpendicular. This structure can provide structural stability to the head 510, improving resistance to deformation against external forces. Therefore, when pulling the head 510 of the medical bone screw 500 inserted into bone in a direction parallel to the central axis C2 of the body 530 using a medical tool, the head 510 does not easily deform, and the pulling force can be stably transmitted to the body 530 through the head 510.

The body 530 includes a first spiral 531 connected to the neck 520 and a second spiral 532 connected to the first spiral 531. The tip 540 is positioned at the end of the second spiral 532. The first spiral 531 and the second spiral 532 can be smoothly surface-treated considering direct contact with bone tissue.

The cross-sectional shape of the first spiral 531 and the second spiral 532 can be circular like the head 510 and the neck 520, but is not limited thereto.

The tip 540 is positioned at the end of the second spiral 532.

The medical bone screw 500 according to yet another embodiment of the present invention includes a central space 550 and an intermediate space 555 defined by the body 530.

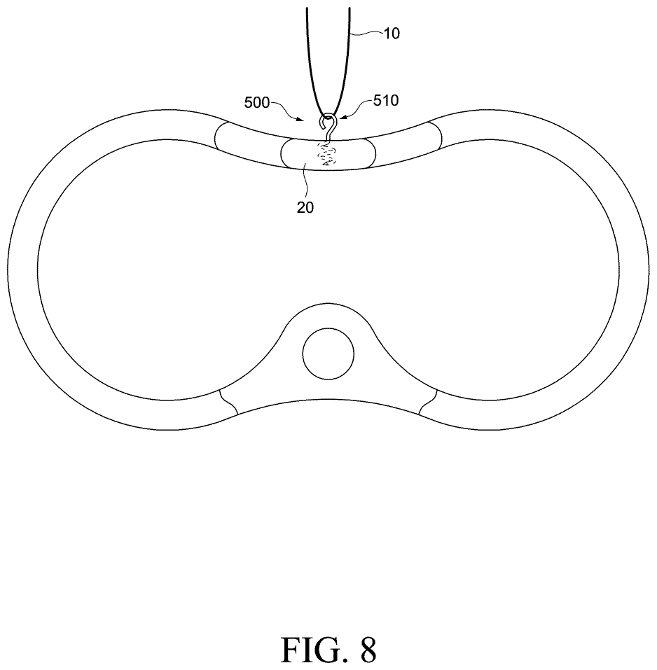

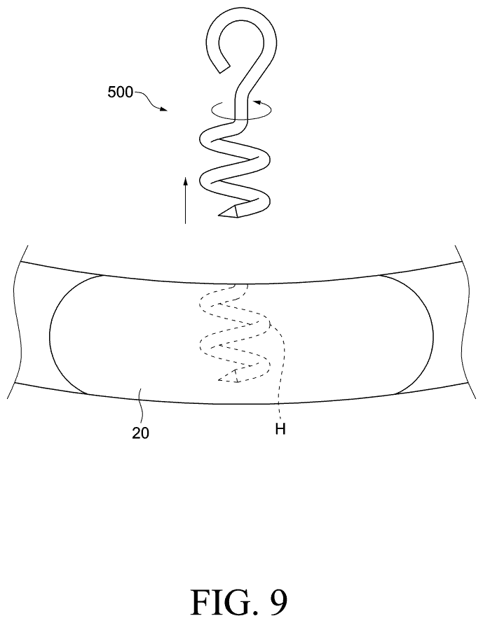

FIG. 8 schematically shows the medical bone screw shown in FIG. 6 inserted into bone tissue, and FIG. 9 schematically shows the medical bone screw shown in FIG. 6 separated from bone tissue.

As shown in FIGS. 8 and 9, the medical bone screw 500 according to yet another embodiment of the present invention can be fixed to bone tissue 20 of the human body. The tip 540 can penetrate the bone tissue 20 by placing the tip 540 against the bone tissue 20 and rotating the head 510 while pressing it using a medical tool such as a medical driver. At this time, the tip 540 and the body 530 enter the bone tissue 20 while forming a spiral hole H in the bone tissue 20.

As shown in FIGS. 8 and 9, the bone tissue 20 that is the insertion target of the medical bone screw 500 can be the sternum, and the medical bone screw 500 can be used for pectus excavatum treatment (Ravitch procedure, Nuss procedure). In this case, the medical bone screw 500 is inserted into the sternum, and by connecting a medical tool 10 such as a medical wire to the head 510 and pulling it, the depressed sternum can be pulled in the outward direction of the chest.

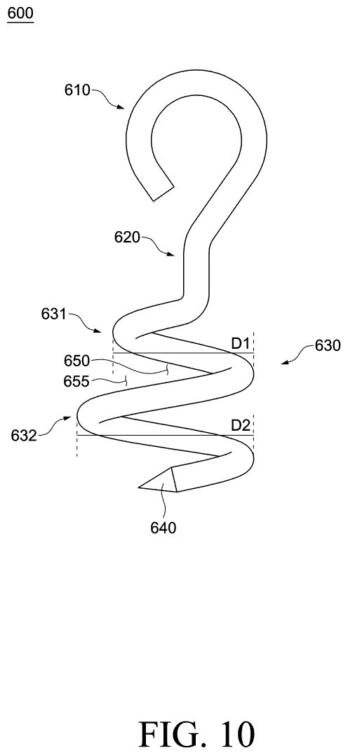

FIG. 10 is a front view showing a medical bone screw according to yet another embodiment of the present invention.

As shown in the drawing, the medical bone screw 600 according to yet another embodiment of the present invention includes a head 610, a neck 620 connected to the head 610, a body 630 connected to the neck 620, a tip 640 disposed at the end of the body 630, and a central space 650 and an intermediate space 655 defined by the body 630. The neck 620 and the body 630 constitute an anchor unit.

The body 630 includes a plurality of spirals 631, 632 connected in sequence. The plurality of spirals 631, 632 includes a first spiral 631 connected to the neck 620 and a second spiral 632 connected to the first spiral 631.

The tip 640 is positioned at the end of the second spiral 632.

The first diameter D1 of the first spiral 631 and the second diameter D2 of the second spiral 632 are different. Specifically, the second diameter D2 of the second spiral 632 is larger than the first diameter D1 of the first spiral 631.

The medical bone screw 600 according to yet another embodiment of the present invention can provide greater fixation strength as the second spiral 632, which is inserted relatively deep into the bone tissue, has a larger contact area with the bone tissue. In particular, it can provide stable fixation strength even in areas with weak bone quality.

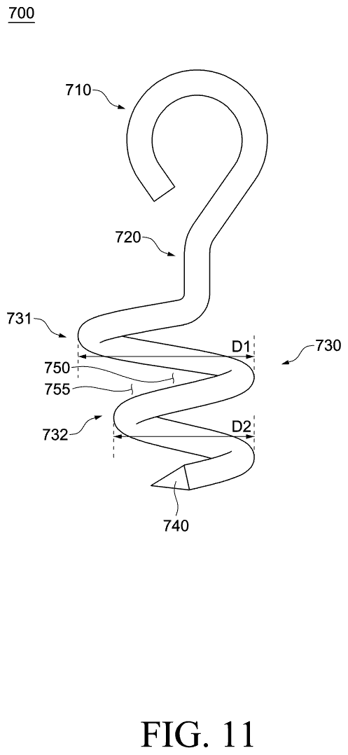

FIG. 11 is a front view showing a medical bone screw according to yet another embodiment of the present invention.

As shown in the drawing, the medical bone screw 700 according to yet another embodiment of the present invention includes a head 710, a neck 720 connected to the head 710, a body 730 connected to the neck 720, a tip 740 disposed at the end of the body 730, and a central space 750 and an intermediate space 755 defined by the body 730. The neck 720 and the body 730 constitute an anchor unit.

The body 730 includes a plurality of spirals 731, 732 connected in sequence. The plurality of spirals 731, 732 includes a first spiral 731 connected to the neck 720 and a second spiral 732 connected to the first spiral 731.

The tip 740 is positioned at the end of the second spiral 732.

The first diameter D1 of the first spiral 731 and the second diameter D2 of the second spiral 732 are different. Specifically, the second diameter D2 of the second spiral 732 is smaller than the first diameter D1 of the first spiral 731.

The medical bone screw 700 according to yet another embodiment of the present invention allows for easier insertion when the second spiral 732 is first inserted into bone tissue due to its relatively smaller diameter, and the subsequently inserted first spiral 731 can provide strong fixation strength as the contact area with bone tissue increases due to its larger diameter. This gradual diameter increase structure has the advantage of securing solid fixation strength while reducing the rotational force required during insertion.

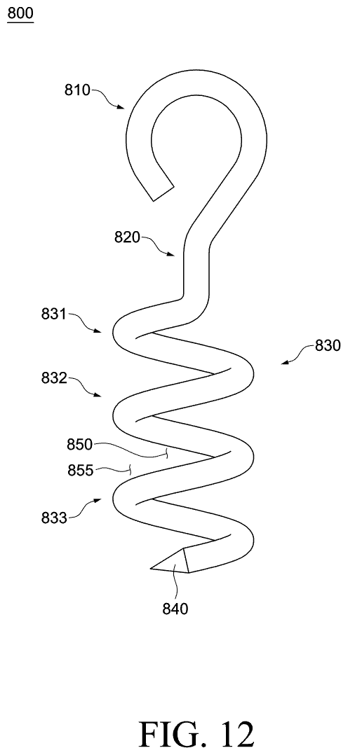

FIG. 12 is a front view showing a medical bone screw according to yet another embodiment of the present invention.

As shown in the drawing, the medical bone screw 800 according to yet another embodiment of the present invention includes a head 810, a neck 820 connected to the head 810, a body 830 connected to the neck 820, a tip 840 disposed at the end of the body 830, and a central space 850 and an intermediate space 855 defined by the body 830. The neck 820 and the body 830 constitute an anchor unit.

The body 830 includes a plurality of spirals 831, 832, 833 connected in sequence. The plurality of spirals 831, 832, 833 includes a first spiral 831 connected to the neck 820, a second spiral 832 connected to the first spiral 831, and a third spiral 833 connected to the second spiral 832.

The tip 840 is positioned at the end of the third spiral 833.

The diameter, pitch, and thickness of each of the first spiral 831, the second spiral 832, and the third spiral 833 can all be the same, but are not limited thereto.

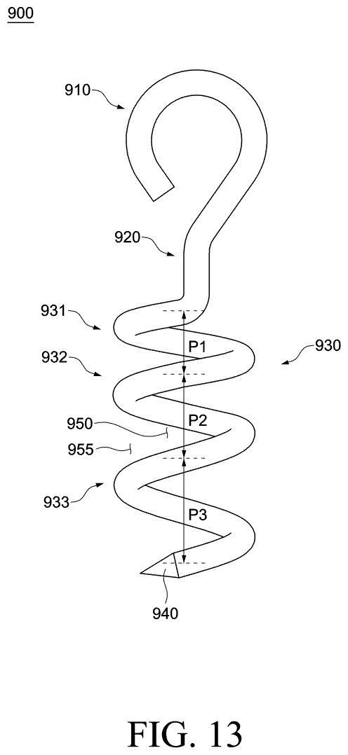

FIG. 13 is a front view showing a medical bone screw according to yet another embodiment of the present invention.

As shown in the drawing, the medical bone screw 900 according to yet another embodiment of the present invention includes a head 910, a neck 920 connected to the head 910, a body 930 connected to the neck 920, a tip 940 disposed at the end of the body 930, and a central space 950 and an intermediate space 955 defined by the body 930. The neck 920 and the body 930 constitute an anchor unit.

The body 930 includes a plurality of spirals 931, 932, 933 connected in sequence. The plurality of spirals 931, 932, 933 includes a first spiral 931 connected to the neck 920, a second spiral 932 connected to the first spiral 931, and a third spiral 933 connected to the second spiral 932.

The tip 940 is positioned at the end of the third spiral 933.

The pitch of each of the first spiral 931, the second spiral 932, and the third spiral 933 is different.

Specifically, the first pitch P1 of the first spiral 931 connected to the neck 920 is smaller than the third pitch P3 of the third spiral 933 connected to the tip 940. The second pitch P2 of the second spiral 932 can be larger than the first pitch P1 and smaller than the third pitch P3.

The medical bone screw 900 according to yet another embodiment of the present invention allows for quick initial entry as the pitch P1, P2, P3 of the spirals 931, 932, 933 gradually decreases in the order of insertion into bone tissue. In addition, stable fixation strength can be secured as the spiral structure becomes more dense as insertion progresses. This gradual pitch change has the effect of optimizing both insertion efficiency and fixation strength.

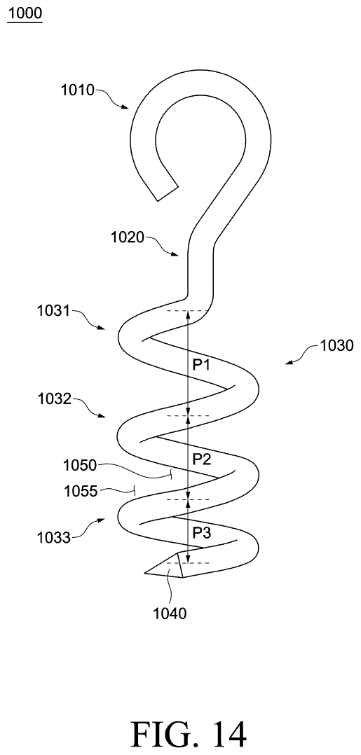

FIG. 14 is a front view showing a medical bone screw according to yet another embodiment of the present invention.

As shown in the drawing, the medical bone screw 1000 according to yet another embodiment of the present invention includes a head 1010, a neck 1020 connected to the head 1010, a body 1030 connected to the neck 1020, a tip 1040 disposed at the end of the body 1030, and a central space 1050 and an intermediate space 1055 defined by the body 1030. The neck 1020 and the body 1030 constitute an anchor unit.

The body 1030 includes a plurality of spirals 1031, 1032, 1033 connected in sequence. The plurality of spirals 1031, 1032, 1033 includes a first spiral 1031 connected to the neck 1020, a second spiral 1032 connected to the first spiral 1031, and a third spiral 1033 connected to the second spiral 1032.

The tip 1040 is positioned at the end of the third spiral 1033.

The pitch of each of the first spiral 1031, the second spiral 1032, and the third spiral 1033 is different.

Specifically, the first pitch P1 of the first spiral 1031 connected to the neck 1020 is larger than the third pitch P3 of the third spiral 1033 connected to the tip 1040. The second pitch P2 of the second spiral 1032 can be smaller than the first pitch P1 and larger than the third pitch P3.

The medical bone screw 1000 according to yet another embodiment of the present invention can increase the contact area in a relatively deep part of the bone tissue and improve fixation strength due to the dense spiral structure resulting from the small pitch of the third spiral 1033, which is inserted relatively deeply into the bone tissue among the body 1030.

The exemplary embodiments of the present disclosure may also be described as follows.

A medical bone screw according to an embodiment of the present invention includes: a head; a body connected to the head and having a spiral shape with dimensions appropriate for medical bone screw applications; and a central space defined by the body.

The body may include a plurality of spirals connected in sequence.

Among the plurality of spirals, the diameter of a spiral relatively closer to the head may be smaller than the diameter of a spiral relatively farther from the head, or the diameter of a spiral relatively closer to the head may be larger than the diameter of a spiral relatively farther from the head, or the pitch of a spiral relatively closer to the head may be smaller than the pitch of a spiral relatively farther from the head, or the pitch of a spiral relatively closer to the head may be larger than the pitch of a spiral relatively farther from the head.

The medical bone screw according to an embodiment of the present invention may further include a neck having a straight shape to connect the head and the body.

The head may include a ring connected to the neck to allow connection of a medical tool.

The medical bone screw according to an embodiment of the present invention may further include a tip disposed at the end of the body to penetrate bone tissue.

The tip may be made in a non-cutting edge form.

The head may include a coupling groove that allows a medical connecting member to be coupled.

The head and the body may be connected to be mutually angle-adjustable.

The medical bone screw according to an embodiment of the present invention may further include a head protrusion protruding from the head to be detachably coupled with a medical tool.

The head may include a coupling groove into which a medical abutment can be inserted.

A medical bone screw according to another embodiment of the present invention includes: a head that can be coupled with a medical tool; and an anchor unit having a first portion extending from the head, and a second portion having a spiral shape extending from the first portion to be inserted into a patient's bone to accommodate bone tissue.

The first portion may have a straight shape.

The first portion may extend from the head with substantially the same cross-sectional area as the head.

The head may have a hook shape, and the central axis of the coupling hole formed by the hook shape and the central axis of the spiral shape may be substantially perpendicular.

A medical medium for human tissue attachment according to an embodiment of the present invention includes: a body having an open spiral shape to penetrate human tissue; and a tissue accommodation space defined by the open spiral shape and configured to improve fixation strength with respect to the human tissue attachment when compared to a medical medium that lacks the tissue accommodation space.

The body may include a plurality of spirals connected in sequence, and the tissue accommodation space may include a central space inside the plurality of spirals and an intermediate space between the plurality of spirals.

The open spiral shape and the tissue accommodation space may be configured to have: a first set of dimensions, with windings of the open spiral shape being relatively tight and the tissue accommodation space being relatively small, which are appropriate for a first type of human tissue or a second set of dimensions, with windings of the open spiral shape being relatively sparse and the tissue accommodation space being relatively large, which are appropriate for a second type of human tissue.

The first type of human tissue may be bone or bone-like material.

The second type of human tissue may be non-bone or non-bone-like material.

Although preferred examples of the present invention have been described above, the scope of the present invention is not limited to the forms described and illustrated above.

For example, while the medical bone screw according to the present invention has been described as being fixed to bone tissue, the screw of the present invention can be used to fix or connect human tissues other than bone tissue.

While the embodiments of this specification have been described in more detail with reference to the attached drawings, this specification is not necessarily limited to such embodiments and can be variously modified and implemented within the scope that does not deviate from the technical spirit of this specification. Therefore, the embodiments disclosed in this specification are not intended to limit the technical spirit of this specification but to explain it, and the scope of the technical spirit of this specification is not limited by these embodiments. Therefore, it should be understood that the embodiments described above are illustrative in all aspects and not restrictive.

What is claimed is:

1. A medical bone screw comprising:
a head;
a body connected to the head and having a spiral shape with dimensions appropriate for medical bone screw applications; and
a central space defined by the body.

2. The medical bone screw of claim 1, wherein the body includes a plurality of spirals connected in sequence.

3. The medical bone screw of claim 2, wherein among the plurality of spirals, the diameter of a spiral relatively closer to the head is smaller than the diameter of a spiral relatively farther from the head, or the diameter of a spiral relatively closer to the head is larger than the diameter of a spiral relatively farther from the head, or the pitch of a spiral relatively closer to the head is smaller than the pitch of a spiral relatively farther from the head, or the pitch of a spiral relatively closer to the head is larger than the pitch of a spiral relatively farther from the head.

4. The medical bone screw of claim 1, further comprising a neck having a straight shape to connect the head and the body.

5. The medical bone screw of claim 4, wherein the head includes a ring connected to the neck to allow connection of a medical tool.

6. The medical bone screw of claim 1, further comprising a tip disposed at the end of the body to penetrate bone tissue.

7. The medical bone screw of claim 6, wherein the tip is made in a non-cutting edge form.

8. The medical bone screw of claim 1, wherein the head includes a coupling groove that allows a medical connecting member to be coupled.

9. The medical bone screw of claim 8, wherein the head and the body are connected to be mutually angle-adjustable.

10. The medical bone screw of claim 1, further comprising a head protrusion protruding from the head to be detachably coupled with a medical tool.

11. The medical bone screw of claim 1, wherein the head includes a coupling groove into which a medical abutment can be inserted.

12. A medical bone screw comprising:
a head that can be coupled with a medical tool; and
an anchor unit having a first portion extending from the head, and a second portion having a spiral shape extending from the first portion to be inserted into a patient's bone to accommodate bone tissue.

13. The medical bone screw of claim 12, wherein the first portion has a straight shape.

14. The medical bone screw of claim 12, wherein the first portion extends from the head with substantially the same cross-sectional area as the head.

15. The medical bone screw of claim 12, wherein the head has a hook shape, and the central axis of the coupling hole formed by the hook shape and the central axis of the spiral shape are substantially perpendicular.

16. A medical medium for human tissue attachment, the medical medium comprising:
a body having an open spiral shape to penetrate human tissue; and
a tissue accommodation space defined by the open spiral shape and configured to improve fixation strength with respect to the human tissue attachment when compared to a medical medium that lacks the tissue accommodation space.

17. The medical medium of claim 16, wherein the body includes a plurality of spirals connected in sequence, and
wherein the tissue accommodation space includes a central space inside the plurality of spirals and an intermediate space between the plurality of spirals.

18. The medical medium of claim 16, wherein the open spiral shape and the tissue accommodation space are configured to have:
a first set of dimensions, with windings of the open spiral shape being relatively tight and the tissue accommodation space being relatively small, which are appropriate for a first type of human tissue or
a second set of dimensions, with windings of the open spiral shape being relatively sparse and the tissue accommodation space being relatively large, which are appropriate for a second type of human tissue.

19. The medical medium of claim 18, wherein the first type of human tissue is bone or bone-like material.

20. The medical medium of claim 18, wherein the second type of human tissue is non-bone or non-bone-like material.
